(19) United States
(12) Patent Application Publication
(10) Pub. No.: US 2022/0229078 A1
(43) Pub. Date: Jul. 21, 2022

(54) OVERACTIVE BLADDER DIAGNOSTIC APPARATUS AND METHODS

(71) Applicant: UNIVERSITY HOSPITALS - CLEVELAND MEDICAL CENTER, Cleveland, OH (US)

(72) Inventors: David Sheyn, Cleveland, OH (US); Adonis Hijaz, Cleveland, OH (US); Sangeeta Mahajan, Cleveland, OH (US)

(21) Appl. No.: 17/577,066

(22) Filed: Jan. 17, 2022

Related U.S. Application Data
(60) Provisional application No. 63/137,947, filed on Jan. 15, 2021.

(51) Int. Cl. G01N 33/94; A61B 5/20; A61B 10/00; G01N 33/543

(52) U.S. Cl. CPC G01N 33/944; A61B 5/202; G01N 33/54387; A61B 10/007; A61B 5/207

(57) ABSTRACT

A method of diagnosing the risk that a subject has or has an increased risk of developing overactive bladder syndrome is described. The method includes detecting the levels of choline and/or acetylcholine in a biological sample from the subject, comparing the detected levels to reference values, and characterizing the subject as having an increased risk of having or developing overactive bladder syndrome if the choline and/or acetylcholine values are higher than the reference values. Kits for determining choline and/or acetylcholine levels in a subject, and methods of treating a subject for overactive bladder syndrome are also described.

### CONTINUING APPLICATION DATA

This application claims the benefit of U.S. Provisional Application Ser. No. 63/137,947, filed Jan. 15, 2021, the disclosure of which is incorporated by reference herein.

### BACKGROUND

Overactive bladder with urinary urgency incontinence (UUI), is estimated to affect millions of women globally; in a national survey of 5,000 households, 9.3% of all women, and 19.1% of those older than 65 years complained of UUI. Women aged 60 to 80 years are among the fastest growing segment of the female population, and thus the prevalence and economic burden of UUI, estimated at nearly $25 billion annually, is projected to rapidly increase over time. Additionally, not only does UUI has a significant negative impact on quality of life (QOL), but is associated with a higher burden and prevalence of anxiety and depression compared to similar groups without lower urinary tract symptoms.

While some patients with UUI have an identifiable underlying cause such a neurodegenerative disease, dementia, pelvic floor prolapse, outlet obstruction and medications, in the majority of cases its etiology is unknown and classified as “idiopathic”. Peyronnet et al., Eur Urol., 75(6):988-1000 (2019). However, accumulating evidence now suggests that idiopathic UUI is likely due to sub-clinical dysfunction of a variety of organ systems including the central, peripheral and autonomic nervous systems, cardiovascular system, endocrine system and musculoskeletal system. Araklitis et al., F1000Res., 11;9:F1000 Faculty Rev-1125 (2020). However, regardless of the underlying causes, the same UUI care-pathway consisting of behavioral modification, pelvic floor physical therapy, pharmacotherapy and third-line therapy, is universally applied to patients presenting with UUI, due to the difficulty discerning the dominant cause for their complaints. This often leads to patient frustration and attrition.

As a result, increased attention is being focused on how to best identify the most suitable treatment for individual patients, using a patient-centered, rather than a “one size fits all” approach. Researchers are exploring utilizing specific biomarkers including interleukins, tumor necrosis factor-alpha, and nerve growth factor to guide therapeutic intervention; however, none of these biomarkers take into consideration the fact that the primary pharmacologic mediators of OAB, which are anti-cholinergic medications. Antunes-Lopes T, Cruz F. Eur Urol Focus., 5(3):329-336 (2019). Previously, the inventors investigated the relationship between choline (Ch) and acetylcholine (Ach) in patients with and without UUI, as well as in patients who responded anti-cholinergic therapy versus those who did not; we identified that concentrations of Ch differed between patient with and without UUI but not between responders and non-responders, and that patients who responded to anti-cholinergic therapy had higher levels of Ach. Sheyn et al., Female Pelvic Med Reconstr Surg., 26(12):e91-e96 (2020); Sheyn et al., Female Pelvic Med Reconstr Surg., 26(10):644-648 (2020) While those studies demonstrated statistically significant findings, they had several limitations, including the inability to assess Ach in patients without UUI due to lack of sensitive assay and a small sample size, and no evaluation of acetylcholinesterase (AchE) activity. Accordingly, there remains a need for an effective method of segregating responders and non-responders before treating patients with anti-cholinergic therapy.

### SUMMARY OF THE INVENTION

In one aspect, the present invention provides a method of diagnosing the risk that a subject has or has an increased risk of developing overactive bladder syndrome, comprising detecting the levels of choline and/or acetylcholine in a biological sample from the subject, comparing the detected levels to control values, and characterizing the subject as having an increased risk of having or developing overactive bladder syndrome if the choline and/or acetylcholine values are higher than the control values.

In some embodiments, the subject is a human female, while in further embodiments, the human female is post-menopausal. In some embodiments, biological sample is urine, while in further embodiments the method further comprising obtaining the biological sample from a subject.

A further aspect of the invention provides a method of treating a subject for overactive bladder syndrome, comprising the step of determining the level of acetylcholine in a biological sample from a subject having overactive bladder syndrome, and treating the subject with a therapeutically effective amount of an anticholinergic agent if the biological sample from the subject is has a higher acetylcholine level than a control value.

In some embodiments, the subject is a human female, while in further embodiments, the human female is post-menopausal. In some embodiments, biological sample is urine, while in further embodiments the method further comprising obtaining the biological sample from a subject. In an additional embodiment, the overactive bladder syndrome is treated using anti-cholinergic therapy.

A further aspect of the invention provides a kit for determining choline and/or acetylcholine levels in a subject, comprising a reagent for detecting the level of choline and/or acetylcholine in a biological sample obtained from the subject, and means for signaling the levels of choline and/or acetylcholine detected in the biological sample.

In some embodiments, the biological sample is urine. In additional embodiments, the kit provides a point-of-care diagnosis. In further embodiments, the reagent for detecting the level of choline and/or acetylcholine is on a dip stick. In some embodiments, the kit only detects acetylcholine. In additional embodiments, the kit further comprises instructions for using the kit to treat or diagnose a subject for overactive bladder syndrome. In yet further embodiments, the kit comprises a package to hold the components of the kit.

In some aspects, the invention will allow clinicians to perform point of care and/or rapid laboratory testing to identify subjects eligible for anti-cholinergic therapy as well those who are likely to not respond to medications and triage those subjects to more appropriate care or at least use that information for more appropriate counseling. It will provide an improved capability for the diagnosis and treatment of lower urinary tract symptoms.

### BRIEF DESCRIPTION OF THE FIGURES

The present invention may be more readily understood by reference to the following figure, wherein:

FIG. 1 provides a flow chart of patient inclusion and exclusion criteria.

### DETAILED DESCRIPTION OF THE INVENTION

The present invention provides a method of diagnosing the risk that a subject has overactive bladder syndrome. The method includes detecting the levels of choline and/or acetylcholine in a biological sample from the subject, comparing the detected levels to reference values, and characterizing the subject as having an increased risk of having or developing overactive bladder syndrome if the choline and/or acetylcholine values are higher than the reference values. In other aspects, the present invention provides kits for determining choline and/or acetylcholine levels in a subject, and methods of treating a subject for overactive bladder syndrome.

### Definitions

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen), and the like.

The term “subject,” as used herein, refers to human or non-human mammal, e.g., a dog, a cat, a mouse, a rat, a cow, a sheep, a pig, a goat, or a primate, and expressly includes laboratory mammals, livestock, pets, and domestic mammals. Preferably, the subject is human Subjects can also be selected from different age groups. For example, the subject can be a child, adult, or elderly subject. The subject can also be male or female, and if female, can be pre- or post-menopausal.

### Diagnosing Overactive Bladder Syndrome

In one aspect, the present invention provides a method of diagnosing the risk that a subject has or has an increased risk of developing overactive bladder syndrome, comprising detecting the levels of choline and/or acetylcholine in a biological sample from the subject, comparing the detected levels to reference values, and characterizing the subject as having an increased risk of having or developing overactive bladder syndrome if the choline and/or acetylcholine values are higher than the reference values. In some embodiments, the subject is a human female, while in further embodiments the human female is post-menopausal.

Overactive bladder syndrome is a condition where there is a frequent feeling of needing to urinate to a degree that it negatively affects a subject's life. The frequent need to urinate may occur during the day, at night, or both. If there is loss of bladder control then it is known as urge incontinence. Overactive bladder is characterized by a group of four symptoms: urgency, urinary frequency, nocturia, and urge incontinence. Diagnosis of overactive bladder syndrome is made primarily on the subject's signs and symptoms and by ruling out other possible causes such as an infection.

As used herein, the term “biological sample” means sample material derived from or contacted by living cells. The term “biological sample” is intended to include tissues, cells and biological fluids isolated from a subject, as well as tissues, cells and fluids present within a subject. Biological samples include, e.g., but are not limited to, whole blood, plasma, serum, semen, cell lysates, saliva, tears, urine, fecal material, sweat, buccal, skin, synovial fluid, cerebrospinal fluid, and hair. Biological samples can also be obtained from biopsies of internal organs. A preferred biological sample for the present invention is urine.

The method can also include the step of the step of obtaining a biological sample from a subject. Alternately, in some embodiments, the biological samples may have already been obtained. Biological samples can be obtained from subjects for diagnosis prognosis, monitoring, or a combination thereof, or for research, or can be obtained from un-diseased individuals, as controls or for basic research. Biological samples can be obtained by any known means including catheter, needle biopsy, swab, and the like.

A biological sample may be fresh or stored. Samples can be stored for varying amounts of time, such as being stored for an hour, a day, a week, a month, or more than a month. The biological sample may be a bodily fluid expressly obtained for the assays of this invention or a bodily fluid obtained for another purpose which can be sub-sampled for the assays of this invention.

The present invention includes a method of detecting choline and/or acetylcholine in a biological sample. For example, the chemical reaction used for detection can correspond to that used in existing urine dipsticks, urinalysis or urine pregnancy tests, Methods of detecting choline are known to those skilled in the art. For example, the amount of choline can be detected in a biological sample by contacting the sample with choline oxidase, and determining the amount of hydrogen peroxide formed. See for example the Total Choline Assay Kit (ab219944) provided by ABCAM™, in which the amount of hydrogen peroxide is detected using a fluorescent red dye. Alternately, choline can be oxidized to form betaine, generating reaction products which react with a choline probe to generate color. See for example the Choline/Acetylcholine quantification kit provided by LSBio™. Choline can also be detected using a portable amperometric ion sensor. Xie et al., ACS Sens., 2, 6, 803-809 (2017). Alternately, choline and acetylcholine can be detected using HPLC. Damsma et al., J Neurochem., 45(5):1649-52 (1985). In some embodiments, acetylcholine can be detected by the same methods used to detect choline by converting acetylcholine to choline using acetylcholinesterase. Preferably, the method provides a quantitative assessment of the level of choline and/or acetylcholine.

The method can be used to detect choline and/or acetylcholine in any suitable biological sample. In some embodiments, the biological sample is obtained from a healthy subject. In other embodiments, the biological sample is obtained from a subject having a disease or disorder. For example, in some embodiments, the subject has been diagnosed as having a urinary tract disorder.

An increased level of choline and/or acetylcholine, as compared with a reference level indicates that the subject has or has an increased chance of developing overactive bladder syndrome. As used herein, the term “reference level” is intended to mean a control level of choline and/or acetylcholine from a healthy individual, or the median value obtained from a number of healthy subjects.

The method can also include the step of providing a report indicating the subject has a overactive bladder syndrome. For example, the method of diagnosis can include the use of a processor coupled to the choline and/or acetylcholine detection assay and adapted to quantify the data representing the signals from the assay, and adapted to perform the multivariate statistical analysis, compare the output value to the first reference value and the second reference value, and calculate the level of choline and/or acetylcholine; and an output display coupled to the processor and configured to report the level of choline and/or acetylcholine, and/or whether the subject has or has an increased risk of developing overactive bladder syndrome. Subjects having higher levels of choline and/or acetylcholine compared with reference levels have an increased likelihood of having or developing overactive bladder syndrome.

In some embodiments, the method of diagnosing overactive bladder syndrome in a subject is carried out on a subject who has been characterized as having an increased risk of having overactive bladder syndrome. For example, in some embodiments, the subject may have risk factors such as overactive bladder syndrome such as aging, female gender, an enlarged prostate, constipation, high caffeine use, or diabetes, or has exhibited symptoms of overactive bladder syndrome such as incontinence. In further embodiments, the subject may have a family history of overactive bladder syndrome.

### Methods of Treating Overactive Bladder Syndrome

In some embodiments, the method of diagnosis is used to guide treatment of subjects identified as having overactive bladder syndrome. In one aspect, the invention provides a method of treating a subject for overactive bladder syndrome that includes the step of determining the level of acetylcholine in a biological sample from a subject having overactive bladder syndrome, and treating the subject with a therapeutically effective amount of an anticholinergic agent if the biological sample from the subject is has a higher acetylcholine level than a reference value. In some embodiments, the subject is a human female, while in further embodiments the human female is post-menopausal. The method can use any of the methods of detecting acetylcholine described herein.

A variety of methods for treating overactive bladder syndrome are known to those skilled in the art. Methods of treatment include lifestyle modifications, such as fluid restriction, avoidance of caffeine, or timed voiding, pelvic floor muscle exercise, use of a neuromodulation device, Botulinum Toxin A injections, administration of antimuscarinic drugs, or anti-cholinergic therapy.

In some embodiments, the overactive bladder syndrome is treated using anti-cholinergic therapy. Anti-cholinergic therapy is therapy directed to blocking the action of acetylcholine. In some embodiments, anti-cholinergic therapy involves administration of an anti-cholinergic agent. Examples of anti-cholinergic agents include atropine, belladonna alkaloids, benztropine mesylate, clidinium, cyclopentolate, darifenacin, dicylomine, fesoterodine, flavoxate, glycopyrrolate, homatropine hydrobromide, hyoscyamine, ipratropium, orphenadrine, oxybutynin, propantheline, scopolamine, methscopolamine, solifenacin, tiotropium, tolterodine, trihexyphenidyl, and trospium.

Another aspect of the invention provides a method of monitoring choline and/or acetylcholine levels in a subject undergoing treatment of overactive bladder syndrome, comprising obtaining a biological sample from the subject, detecting choline and/or acetylcholine levels in the biological sample, comparing the detected choline and/or acetylcholine levels with the choline and/or acetylcholine level reference values, and altering the therapy to increase the level(s) of choline and/or acetylcholine in the subject if the detected choline and/or acetylcholine levels are lower than the corresponding reference values.

### Kits

Another aspect of the present invention provides a kit for determining choline and/or acetylcholine levels in a subject. The kit includes a reagent for detecting the level of choline and/or acetylcholine in a biological sample obtained from the subject, and means for signaling the levels of choline and/or acetylcholine detected in the biological sample. In some embodiments, the biological sample is urine. In some embodiments, the kit detects choline, in some embodiments the kit detects acetylcholine, and in some embodiments the kit detects choline and acetylcholine.

In accordance with another embodiment, the present invention provides one or more kits for determining the level of choline and/or acetylcholine in a subject, diagnosing a subject has having overactive bladder syndrome, or monitoring treatment of overactive bladder syndrome in a subject. The kits components of the kits will vary depending on whether they are intended for evaluation, diagnosis, or monitoring. The kit should include one or more chemicals capable of detecting choline and/or acetylcholine, and signaling that detection to a user of the kit. The kit also includes reagents, buffers, and the like for carrying out an assay, which are known to those of ordinary skill in the art.

Components of the kits of the present invention may be in different physical states. For example, some components may be lyophilized and some in aqueous solution. Some may be frozen. Individual components may be separately packaged within the kit. Other useful tools for performing the methods of the invention or associated testing, therapy, or calibration may also be included in the kits, including buffers, enzymes, chemiluminescence reagents, PMAT reagents, gels, plates, detectable labels, vessels, etc. Kits may also include a sampling device for obtaining a biological sample from a subject, such as a syringe or needle, a urine collection cup, or a dipstick including the reagent for detecting the level of choline and/or acetylcholine.

In some embodiments, the kit provides a point-of-care diagnosis. Point-of-care diagnosis is a diagnosis that can be provided at the same time that a clinician is conducting the assay for the subject (e.g., a human patient). Accordingly, a point-of-care kit should include all of the components necessary to detect choline and/or acetylcholine, a reference level to compare the detected levels to, and a guide that indicates when a subject should be diagnosed with overactive bladder syndrome based on the detected levels. In addition, the point-of-care kit should be capable of providing results quickly, and being provided in a relatively non-bulky package for ease of use.

In some embodiments, the kit includes a package to hold the components of the kit. The package contains all elements of the kit, such as the chemicals for detection and the optional instructions, The package may be divided so that components are not mixed until desired. Kits can be formed using standard packaging materials, such as cardboard or polyurethane. In some embodiments, the kit is transparent so that one can see the components within the kit. The package for the kit can also include a label that characterized the use for the kit.

In some embodiments, the kit includes instructions for using the kit to treat or diagnose a subject for overactive bladder syndrome. Instructions may be in any form, including paper or digital. The instructions describe the procedure for using the kit to detect choline and/or acetylcholine in a biological sample, and then using the detected levels to guide treatment or provide a diagnosis. The instructions may be on the inside or the outside of the package. The instructions may be in the form of an internet address which provides the detailed manipulative or analytic techniques.

Examples have been included to more clearly describe particular embodiments of the invention. However, there are a wide variety of other embodiments within the scope of the present invention, which should not be limited to the particular examples provided herein.

### EXAMPLES

### Example 1: Evaluation of the Relationship of Cholinergic Metabolites in Urine and Urgency Urinary Incontinence

In this study, the inventors measured acetylcholine (Ach), Choline (Ch), and acetylcholinesterase (AchE) differences in a larger group of patients, with and without urinary urgency incontinence UUI, as well as differences in the urinary markers in responders and non-responders to anti-cholinergic therapy.

Materials and Methods

Patients presenting to female pelvic medicine and reconstructive surgery clinics were recruited for study participation if they met the following inclusion criteria: were 18 years or older, with symptoms of urgency incontinence for a minimum of three months and who were eligible for treatment with anticholinergic medications with no prior history of third-line therapy for OAB, current behavioral and/or formal pelvic floor physical therapy, or undergoing treatment with anti-cholinergic or beta-3-agonists within 4 weeks of recruitment. Patients with mixed-urinary incontinence were included if their urgency incontinence was the dominant complaint. Patients performing self-directed Kegel exercises were allowed to participate.

Any woman with the following criteria were excluded from analysis: women who were pregnant within 12 months of study recruitment, currently breast feeding, had a post void residual greater than 200 mL, a history of bladder augmentation, anti-incontinence surgery, a history of an acquired or congenital neurologic disorder known to be associated with voiding dysfunction including but not limited to neural tube defects, multiple sclerosis, traumatic spinal cord or brain injury, myasthenia gravis, and diabetes mellitus; were being treated with non-bladder specific medication that had anticholinergic properties, a diuretic, local or systemic estrogen or selective estrogen receptor modulators; had a history of liver disease, alcohol abuse, narrow-angle glaucoma, chronic kidney disease; were currently being evaluated or treated for recurrent urinary tract infections, interstitial cystitis, chronic pelvic pain and/or dyspareunia, or had a history of genitourinary malignancy. Patients were also excluded if urinalysis data was concerning for urinary tract infection and/or demonstrated hematuria.

If patients met all exclusion and inclusion criteria they were invited to participate in the study and, if they agreed, written informed consent was obtained. Demographic and clinical characteristics were collected for each participant including: age, BMI, parity menopausal status, pelvic surgery history, and smoking status from the electronic medical record and by history. All patients were asked to complete the Overactive Bladder Symptom Score (OABSS), the Urogenital Distress Inventory-6 (UDI-6) and Incontinence Impact Questionnaire-7 (IIQ-7). Homma et al., Urology, 68(2):318-23 (2006). All patients underwent standard pelvic exam as part of the assessment of their presenting complaint, which included pelvic organ prolapse quantification (POP-Q) while in semi-recumbent position and with Valsalva maneuver. Patients also underwent evaluation of a post-void residual by ultrasound, to exclude urinary retention.

Patients were treated for approximately 12 weeks and were contacted by telephone and were again asked to complete IIQ-7, UDI-6, and OABSS questionnaires. Treatment success was defined as at least >50% decrease in OABSS scores. The original study protocol was designed for patients to come to clinic to submit a post-treatment urine sample, however, with the onset of the coronavirus pandemic many patients voiced concern about office visits and shortly before the final group of patients were scheduled to submit samples, elective in person clinic visits were temporarily shut down. As a result, there were insufficient samples to conduct a post-treatment urinary analysis.

### Sample Collection, Storage and Analysis

The protocol for specimen collection is as follows. Specimens were collected mid-stream into 120 mL containers per routine protocol for urinalysis (UA) in the office, and were evaluated for blood, nitrites, moderate or high leukocyte esterase; samples positive for any of these findings were excluded from analysis. To account for differences in urinary dilution, samples that were too dilute, specific gravity (SG) less than 1.015, or too concentrated, SG more than 1.030, were excluded from the study. Samples were stored in a freezer in the clinic at −20 degree Centigrade for a period of 4 to 6 hours and were transported to the proteomics laboratory, and stored at −80 degrees Centigrade. Transit time from the clinic site to the laboratory was under thirty minutes.

The method for evaluating urine Ach and Ch is provided in Example 2, herein. Acetylcholinesterase (AchE) levels were measured using an ELISA kit (Human ACHE ELISA kit, Aviva Systems Biology) and following the manufacturer's protocol. All incubation steps were carried out at 37° C. Briefly, urine samples were thawed, centrifuged 2000 xg for 5 minutes and 100 μL added in duplicate along with a standard curve to microtiter plate wells pre-coated with capture antibody, then incubated for 2 hr. Liquid was removed and 100 μL of biotinylated detector antibody added, followed by incubation for 1 hr. Liquid was again removed and the plate washed 3 times. 100 μL of avidin-horseradish peroxidase conjugate was added to the wells and incubated for 1 hr. After 5 more plate washes, 90 μL of TMB substrate was added and the plate incubated in the dark for 15-30 minutes until the wells turn gradations of blue. Color development was stopped, and changed to yellow, by adding 50 μL of stop solution. O.D. absorbance at 450 nm was read within 5 minutes on a microplate reader (accuSkan FC, Fisher Scientific). AchE was quantified by comparing sample absorbance at 450 nm with that of the standard curve using microplate reader software (SkanIt RE 4.0, Thermo Scientific).

### Statistical Analysis

Based on previous studies, the inventors determined that they would need 14 patients in the responder and non-responder groups to detect statistically significant (p<0.05) differences in acetylcholine and choline levels with 80% power. To account for patient attrition and given a 40-50% treatment failure rate they determined they would need to recruit a total of 40 patients with urgency incontinence. These were matched to patients without urgency incontinence by age. Groups were compared first by presence or absence of UUI, and a sub-group analysis of responders and non-responders was performed. Descriptive statistics are expressed as medians and interquartile ranges. Pairwise analysis using Wilcoxon-Rank Sum test for continuous variables and Fisher's exact test for categorical variables as appropriate to express differences between groups with statistical significance set at p <0.05. Spearman's rho correlation coefficient was used to determine the relationship between Ach, AchE and Ch concentrations and pre- and post-treatment daytime frequency, urgency incontinence episodes, nocturia episodes, OABSS, UDI-6, and IIQ-7 scores; as well as age, BMI, and parity. All statistical analysis was performed using STATA version 14.1 (Stata Corp, College Station, Tex.).

Results

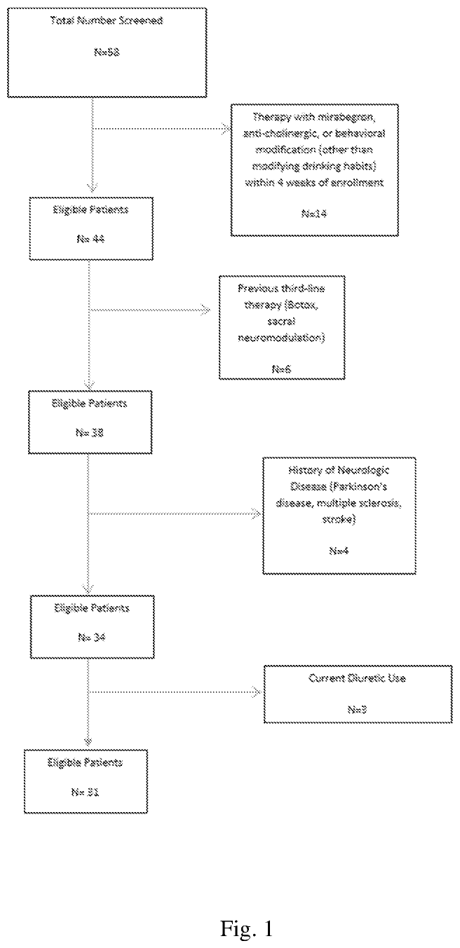

Eighty-nine women were screened during the study period and 72 were enrolled in the study, 39 with urgency incontinence and 33 controls. The most common reason for exclusion was opting for therapy with mirabegron or behavioral modification only; additional reasons for exclusion included abnormal UA findings, previous therapy with third-line therapy for OAB or current diuretic use. A flow chart showing patient inclusion and exclusion criteria is provided in FIG. 1.

Patients with UUI were similar in age to controls, (63 IQR: 52-74 and 64 IQR: 52-77, p=0.96 respectively), and did not differ on the bases of race, smoking status, comorbid stress incontinence, post-void residual and prior pelvic floor surgery, Table 1. Patients with UUI had higher BMI than controls, 33.8 (IQR: 29.2-37.8) kg/m² versus 28.5 (IQR: 25.8-31.2) kg/m², p=0.006; had fewer children, p=0.001, and were less likely to have anterior wall and apical descent, p=0.001 and p=0.008, respectively. For patients with UUI the median pre-treatment OABSS score was 11(IQR: 9-12), the median pre-treatment UDI-6 score was 47.9 (IQR: 37.5-58.0), and the median pre-treatment IIQ-7 score was 60.2 (IQR: 42.8-71.5). The concentrations of Ch 29.0 (IQR: 24.2-42.5) μmol versus 15.2 (IQR: 7.5-24.1) μmol, and acetylcholine 65.8 (IQR: 30.4-101.8) nmol and 33.1 (IQR: 11.9-43.8) nmol, were significantly higher in the UUI group compared to controls; p=0.003 and p<0.001, respectively.

Of the 39 patients with UUI, 43.6% (n=17) responded to medications, after twelve weeks of therapy, and the remainder did not. The responder group had lower BMI, 29.5 kg/m² (IQR: 25.1-33.9) compared to non-responders, 36.0 kg/m² (IQR: 32.1-42.9), p=0.03, Table 2. The groups did not differ in age, parity, smoking history, comorbid stress incontinence, surgical history or POP-Q. The responder group also had higher pre-treatment nocturia scores, with a median 3 or more episodes per night, versus a median of 2 episodes per night for non-responders. Both groups had similar pre-treatment OABSS, UDI-6, and IIQ-7 scores. The most common medication used for treatment of UUI in both groups was oxybutynin, accounting for 66.9%, and the remainder were prescribed trospium. The median number of medications trialed prior to initiation of anti-cholinergic therapy was 0 (IQR: 0-0).

The median time to follow up was approximately 90 days for both groups, p=0.65; the median post-treatment OABBS score in the responder group was 4 (IQR: 2-5) and 11 (IQR: 9-12) in the non-responder group, p<0.001. The median post-treatment UDI-6 score was 30 (IQR:17-52) and 50 (IQR: 42-58), p=0.002, in the responder and non-responder groups, respectively; and the median post-treatment IIQ-7 scores were 49 (IQR:36-61) in the responder group and 65 (IQR: 48-76) in the non-responder group, p=0.01. There was no difference in Ch (p=0.38) or AchE (p=0.85) between groups; acetylcholine levels were higher in the responder group, 82.1 nmol (IQR: 54.8-118.1), compared to the non-responder group, 50.3 nmol (IQR: 29.9-68.2).

Table 3 demonstrates the clinical characteristics and urine metabolite levels in the control group compared to responders and non-responders. Both responders and non-responders had fewer children compared to controls, p=0.01 and p=0.02, respectively. Non-responders had significantly higher BMI compared to controls, 36.0 kg/m² versus 28.5 kg/m² with p=0.001; while BMI was similar between controls and responders. The responder and non-responder groups both were more likely to not have anterior or apical prolapse compared to the control group, where the median value of Aa was −1 and C was −6. The Ch and Ach levels in responders and non-responders were significantly higher than in the control group.

The relationships between Ach and Ch levels and patient characteristics are shown in Table 4. Age was not found to be associated with levels of either metabolite; however, BMI had a weak positive correlation with choline, Spearman p=0.369, p=0.001. Both choline and acetylcholine demonstrated significant but predominantly weak positive associations with pre-treatment frequency, urgency, UUI, and nocturia scores as well as the total OABSS score. The strongest of these associations was between acetylcholine and pre-treatment urgency, Spearman p=0.426, p=0.03.

Discussion:

In this prospective cohort study, the inventors found that women with UUI have significantly higher levels of choline and acetylcholine compared to women without any urgency symptoms; and that women with UUI who respond to anti-cholinergic therapy have higher levels of acetylcholine compared to women who do not respond. No differences in acetylcholinesterase activity was noted. The results add to previous work by the inventors on evaluation of cholinergic urinary metabolites in women with and without UUI and in responders and non-responders.

In the prior study evaluating these metabolites in women with and without UUI, acetylcholine could not be measured due to the lack of a sensitive assay for measuring acetylcholine levels, since then an essay has been acquired with increased sensitivity to Ach allowing these measurements; in this study, Ach levels were found to be higher among women with UUI, as were Ch levels, which differs from our prior results despite using identical measurement technique in both studies. The difference in Ch levels between the two studies may be related differences in the demographics of women, with women in this study being a median of ten years older, and more likely to be post-menopausal. Indeed, there is a negative correlation between Ch and age, indicating that levels may decrease with age, however, this would need to be formally evaluated in a much larger patient cohort, however, this has not been previously investigated.

Acetylcholine is the primary neurotransmitter involved in normal and dysfunctional detrusor contraction, and neuronal acetylcholine has classically been associated with OAB. Yamada et al., Pharmacol Ther., 189:130-148 (2018). However, there is increasing evidence that the urothelium is a metabolically active region of the bladder, capable of producing its own, non-neuronal acetylcholine, and has been implicated in both dysfunction detrusor contraction and as a target of anti-cholinergic medications. Lips et al., Eur Urol., 51(4):1042-53 (2007); Fry C H, Vahabi B., Basic Clin Pharmacol Toxicol., 119 Suppl 3(Suppl 3):57-62 (2016) While in this study the inventors were unable to ascertain whether the acetylcholine measured is neuronal or non-neuronal, the acetylcholine levels were directly related to choline levels, which were elevated in both UUI relative to controls, and in responders relative to non-responders. Choline is an essential nutrient and the primary precursor molecule of acetylcholine, and is not excreted into urine by the kidneys; thus, the choline measured in this and prior studies by the inventors may be directly related to choline being utilized by the urothelium. Ueland P M., J Inherit Metab Dis., 34(1):3-15 (2011).

Additionally, there is an emerging link between microbiome and urinary choline and acetylcholine. Several studies have identified differences in the urinary microbiome and in women with and without UUI, as well as those who do and do not respond to medications. Specifically several studies have found that women with UUI have higher levels of uropathogenic bacteria, and in particular Proteus and Aerococcus species, and lower levels of Lactobacillus species without overt evidence of infection. Wu et al., Front Cell Infect Microbiol., 7:488 (2017) Furthermore, there is evidence that some bacterial species which make up the urinary microbiome can synthesize and release neurotransmitters, including Ach. Cryan J F, Dinan T G, Nat Rev Neurosci., 13(10):701-12 (2012). Thus, the link between UUI and the microbiome could be related to bacterial production of cholinergic metabolites.

The findings of higher acetylcholine levels in responder to anti-cholinergic therapy may suggest that there is a sub-type of OAB, which is primarily mediated by cholinergic signaling mechanisms and may, at least in part, explain why certain patients have dramatic improvements with anti-cholinergic treatment, whereas others with seemingly identical clinical characteristics do not.

This study has several strengths, first, this is the largest cohort to date to investigate differences in cholinergic urine metabolites in women with and without overactive bladder. Second, the inventors were able to reproduce our results with a different study population demonstrating higher levels of acetylcholine and choline in women who respond to anti-cholinergic medications compared to those who did not. They were also able to link choline and acetylcholine levels to OABSS scores, which have been shown to be predictive of treatment response to anti-cholinergic medications. Hsiao et al., Int Neurourol J., 19(3):171-7 (2015)

In summary, this study identified higher levels of choline and acetylcholine in patients with UUI compared to controls, and higher levels of acetylcholine among responders compared to non-responders. Acetylcholine could potentially serve a marker for selection of candidates who may respond to anti-cholinergic therapy, while, patients with low levels may be prescribed other therapies. However, further study in larger cohorts and by different investigator teams is recommended to both confirm and expand on these findings.

TABLE 1
Characteristics of Urinary Urgency Incontinence Patients

| | Urgency | | No Urgency | | |
|---|---|---|---|---|---|
| | Incontinence | | Incontinence | | |
| | N = 39 | | N = 33 | | ^{P} |
| Age (years) | 63 | (52-74) | 64 | (52-77) | 0.96 |
| Body Mass Index (kg/m²) | 33.8 | (29.2-37.8) | 28.5 | (25.8-31.2) | 0.006 |
| Parity | 2 | (1-2) | 3 | (2-4) | 0.001 |
| Non-white Race | 11 | (28.2) | 15 | (45.5) | 0.05 |
| Current Smoker | 8 | (20.5) | 6 | (18.2) | 0.52 |
| Presence of Stress Urinary | 22 | (56.4) | 16 | (48.5) | 0.86 |
| Incontinence | | | | | |
| Constipation | 4 | (10.2) | 3 | (9.1) | 0.62 |
| Post-Void Residual (mL) | 34 | (0-69) | 15 | (0-44) | 0.17 |
| History of Hysterectomy | 10 | (25.6) | 11 | (33.3) | 0.89 |
| Prior History of Pelvic | 5 | (12.8) | 9 | (27.3) | 0.70 |
| Organ Prolapse Repair | 2 | (5.1) | 1 | (3.0) | |
| Apical | 2 | (5.1) | 5 | (15.2) | |
| Anterior | 1 | (2.6) | 3 | (9.1) | |
| Posterior | | | | | |
| Post-Menopausal Status | 28 | (71.8) | 25 | (75.8) | 0.91 |
| Aa | −3 | (−3 to −2) | −1 | (−3 to 1) | 0.001 |
| C | −8 | (−8-6) | −6 | (−8 to −3) | 0.008 |
| Pretreatment OABSS | 1 | (0-2) | 0 | | — |
| Frequency Score | | | | | |
| Pretreatment OABSS | 3 | (2-3) | 0 | | — |
| Nocturia Score | | | | | |
| Pretreatment OABSS | 4 | (3-5) | 0 | | — |
| Urgency Score | | | | | |
| Pretreatment OABSS | 3 | (2-4) | 0 | | — |
| Urgency Incontinence Score | | | | | |
| Pretreatment OABSS Total | 11 | (9-12) | 0 | | — |
| Pretreatment Urogenital | 47.9 | (37.5-58) | — | | — |
| Distress Inventory-6 Score | | | | | |
| Pretreatment Incontinence | 60.2 | (42.8-71.5) | — | | — |
| Impact Questionnaire-7 Score | | | | | |
| Urine Specific Gravity | 1.015 | (1.015-1.020) | 1.020 | (1.015-1.020) | 0.79 |
| Choline (micromoles) | 29.0 | (24.2-42.5) | 15.2 | (7.5-24.1) | 0.003 |
| Acetylcholine (nanomoles) | 65.8 | (30.4-101.8) | 33.1 | (11.9-43.8) | <0.001 |
| Acetylcholinesterase (ng/mL) | 0.14 | (0.06-0.40) | 0.18 | (0.05-0.39) | 0.90 |

TABLE 2
Responder vs. Non-responder characteristics

| | Responder | | Non-Responder | | |
|---|---|---|---|---|---|
| | N = 17 | | N = 22 | | ^{P} |
| Age (years) | 63 | (55-79) | 57 | (47-72) | 0.21 |
| Body Mass Index (kg/m²) | 29.9 | (25.1-33.9) | 36.0 | (32.1-42.9) | 0.03 |
| Parity | 2 | (1-3) | 2 | (1-2) | 0.98 |
| Non-white Race | 5 | (29.4) | 6 | (27.2) | 0.43 |
| Current Smoker | 3 | (17.6) | 5 | (22.7) | 0.51 |
| Presence of Stress | 12 | (70.6) | 10 | (45.4) | 0.11 |
| Urinary Incontinence | | | | | |
| Constipation | 3 | (17.6) | 1 | (4.5) | 0.21 |
| Post-Void Residual (mL) | 17 | (0-37) | 50 | (14-76) | 0.07 |
| History of Hysterectomy | 5 | (29.4) | 5 | (22.7) | 0.45 |
| Prior History of Pelvic | 2 | (11.8) | 3 | (13.6) | 0.69 |
| Organ Prolapse Repair | 1 | (5.9) | 1 | (4.5) | |
| Apical | 1 | (5.9) | 1 | (4.5) | |
| Anterior | 0 | (0.0) | 1 | (4.5) | |
| Posterior | | | | | |
| Post-Menopausal Status | 13 | (76.4) | 15 | (68.2) | 0.30 |
| Aa | −3 | (−3 to −1) | −3 | (−3 to −1) | 0.49 |
| C | −8 | (−8 to −6) | −8 | (−8 to −8) | 0.35 |
| Number of Prior | 0 | (0-0) | 0 | (0-0) | 0.92 |
| Medications Trialed | | | | | |
| Medication Trialed | 12 | (70.1) | 14 | (63.6) | 0.21 |
| Oxybutynin | 5 | (29.4) | 6 | (27.2) | 0.45 |
| Trospium | | | | | |
| Pretreatment OABSS | 1 | (1-2) | 1 | (0-1) | 0.23 |
| Frequency Score | | | | | |
| Pretreatment OABSS | 3 | (3-3) | 2 | (2-3) | 0.03 |
| Nocturia Score | | | | | |
| Pretreatment OABSS | 4 | (4-5) | 4 | (3-4) | 0.79 |
| Urgency Score | | | | | |
| Pretreatment OABSS | 3 | (2-4) | 2 | (1-4) | 0.40 |
| Urgency Incontinence Score | | | | | |
| Pretreatment OABSS Total | 11 | (9-11) | 12 | (9-13) | 0.15 |
| Pretreatment Urogenital | 56 | (42-67) | 50 | (42-58) | 0.09 |
| Distress Inventory-6 Score | | | | | |
| Pretreatment Incontinence | 67 | (54-75) | 69 | (48-81) | 0.13 |
| Impact Questionnaire-7 Score | | | | | |
| Post-treatment OABSS | 0 | (0-0) | 1 | (1-2) | 0.006 |
| Frequency Score | | | | | |
| Post-treatment OABSS | 1 | (0-2) | 2 | (2-3) | 0.001 |
| Nocturia Score | | | | | |
| Post-treatment OABSS | 2 | (1-2) | 3 | (2-4) | 0.001 |
| Urgency Score | | | | | |
| Post-treatment OABSS | 1 | (0-1) | 2 | (2-4) | <0.001 |
| Urgency Incontinence Score | | | | | |
| Post-treatment OABSS Total | 4 | (2-5) | 11 | (9-12) | <0.001 |
| Post-treatment Urogenital | 30 | (17-52) | 44 | (33-51) | 0.002 |
| Distress Inventory-6 Score | | | | | |
| Post-treatment Incontinence | 49 | (36-61) | 65 | (48-76) | 0.01 |
| Impact Questionnaire-7 Score | | | | | |
| Duration of Therapy (days) | 89 | (84-93) | 91 | (84-96) | 0.65 |
| Urine Specific Gravity | 1.020 | (1.015-1.020) | 1.020 | (1.015-1.020) | 0.93 |
| Choline (micromoles) | 37.8 | (14.3-54.1) | 27.0 | (21.7-36.2) | 0.38 |
| Acetylcholine (nanomoles) | 82.1 | (54.8-118.1) | 50.3 | (29.9-68.2) | 0.04 |
| Acetylcholinesterase (ng/mL) | 0.13 | (0.08-0.21) | 0.13 | (0.03-0.40) | 0.85 |

TABLE 3
Clinical Characteristics and Urine Metabolite Levels

| | No Urgency | | Non- | | | | | |
|---|---|---|---|---|---|---|---|---|
| | Incontinence | | Responder | | | Responder | | |
| | N = 33 | | N = 22 | | P* | N = 17 | | p^{!} |
| Age (years), median (IQR) | 64 | (52-77) | 57 | (47-72) | 0.32 | 63 | (55-79) | 0.54 |
| Body Mass Index (kg/m²) | 28.5 | (25.8-31.2) | 36.0 | (32.1-42.9) | 0.001 | 29.9 | (25.1-33.9) | 0.35 |
| Parity | 3 | (2-4) | 2 | (1-2) | 0.02 | 2 | (1-3) | 0.01 |
| Non-white Race | 15 | (45.5) | 6 | (27.2) | 0.18 | 5 | (29.4) | 0.25 |
| Current Smoker | 6 | (18.2) | 5 | (22.7) | 0.68 | 3 | (17.6) | 0.92 |
| Presence of Stress | 16 | (48.5) | 10 | (45.4) | 0.82 | 12 | (70.6) | 0.17 |
| Urinary Incontinence | | | | | | | | |
| Constipation | 3 | (9.1) | 1 | (4.5) | 0.52 | 3 | (17.6) | 0.41 |
| Post-Void Residual (mL) | 15 | (0-44) | 50 | (14-76) | 0.06 | 17 | (0-37) | 0.91 |
| History of Hysterectomy | 11 | (33.3) | 5 | (22.7) | 0.41 | 5 | (29.4) | 0.89 |
| Prior History of Pelvic Organ | 9 | (27.3) | 3 | (13.6) | 0.64 | 2 | (11.8) | 0.58 |
| Prolapse Repair | 1 | (3.0) | 1 | (4.5) | | 1 | (5.9) | |
| Apical | 5 | (15.2) | 1 | (4.5) | | 1 | (5.9) | |
| Anterior | 3 | (9.1) | 1 | (4.5) | | 0 | (0.0) | |
| Posterior | | | | | | | | |
| Post-Menopausal Status | 25 | (75.8) | 15 | (68.2) | 0.54 | 13 | (76.4) | 0.63 |
| Aa | −1 | (−3 to 1) | −3 | (−3 to −1) | 0.002 | −3 | (−3 to −1) | 0.01 |
| C | −6 | (−8 to −3) | −8 | (−8 to −8) | 0.01 | −8 | (−8 to −6) | 0.03 |
| Urine Specific Gravity | 1.020 | (1.015-1.020) | 1.020 | (1.015-1.020) | 0.89 | 1.020 | (1.015-1.020) | 0.92 |
| Choline (micromoles) | 15.2 | (7.5-24.1) | 27.0 | (21.7-36.2) | 0.001 | 37.8 | (14.3-54.1) | 0.002 |
| Acetylcholine (nanomoles) | 33.1 | (11.9-43.8) | 50.3 | (29.9-68.2) | 0.007 | 82.1 | (54.8-118.1) | <0.001 |
| Acetylcholinesterase (ng/mL) | 0.18 | (0.05-0.39) | 0.13 | (0.03-0.40) | 0.85 | 0.13 | (0.08-0.21) | 0.86 |

TABLE 4
Relationships between Ach and Ch levels and Patient Characteristics

| | Spearman's ρ | | Spearman ρ | | Spearman's ρ | |
|---|---|---|---|---|---|---|
| | (Acetylcholi ne) | p | (Choline) | P | (Acetylcholinesterase) | P |
| Age(years) | −0.133 | 0.26 | −0.191 | 0.11 | 0.046 | 0.70 |
| Bodv Mass Index (kg/m2) | 0.192 | 0.11 | 0.369 | 0.001 | −0.048 | 0.69 |
| Parity | −0.150 | 0.21 | −0.137 | 0.25 | −0.021 | 0.86 |
| Pretreatment OABSS | 0.333 | 0.005 | 0.276 | 0.02 | 0.027 | 0.83 |
| Frequency Score | | | | | | |
| Pretreatment OABSS Nocturia | 0.233 | 0.05 | 0.275 | 0.02 | −0.006 | 0.97 |
| Score | | | | | | |
| Pretreatment OABSS Urgency | 0.426 | 0.03 | 0.273 | 0.02 | 0.029 | 0.81 |
| Score | | | | | | |
| Pretreatment OABSS | 0.300 | 0.02 | 0.286 | 0.01 | 0.001 | >0.99 |
| Urgency Incontinence Score | | | | | | |
| Pretreatment OABSS Total | 0.301 | 0.01 | 0.301 | 0.01 | 0.019 | 0.88 |
| Pretreatment Urogential | 0.285 | 0.06 | 0.381 | 0.01 | 0.089 | 0.58 |
| Distress Inventory-6 Score | | | | | | |
| Pretreatment Incontinence | 0.401 | 0.008 | 0.356 | 0.02 | 0.154 | 0.32 |
| Impact Questionnaire-7 Score | | | | | | |
| Post-treatment OABSS | 0.110 | 0.55 | 0.035 | 0.85 | 0.091 | 0.62 |
| Frequency Score | | | | | | |
| Post-treatment OABSS | −0.179 | 0.31 | −0.157 | 0.37 | −0.205 | 0.24 |
| Nocturia Score | | | | | | |
| Post-treatment OABSS | −0.037 | 0.84 | −0.013 | 0.94 | −0.208 | 0.25 |
| Urgency Score | | | | | | |
| Post-treatment OABSS | 0.075 | 0.67 | 0.071 | 0.70 | −0.056 | 0.76 |
| Urgency Incontinence Score | | | | | | |
| Post-treatment OABSS Total | −0.005 | 0.97 | −0.071 | 0.70 | −0.069 | 0.71 |
| Post-treatment Urogential | −0.196 | 0.098 | −0.176 | 0.14 | 0.006 | 0.89 |
| Distress Inventory-6 Score | | | | | | |
| Post-treatment Incontinence | −0.229 | 0.05 | −0.139 | 0.25 | 0.001 | >0.99 |
| Impact Questionnaire-7 Score | | | | | | |

### Example 2: Evaluate of Choline and Acetylcholine Values in Urine Samples

After collection, urine specimens were evaluated by office urinalysis. Specimens that were positive for blood, nitrites, and moderate or high leukocyte esterase were excluded from analysis. In addition, to account for differences in urinary dilution, specimens with specific gravity less than 1.015 and greater than 1.030 were excluded. Previous studies have shown that normalization to specific gravity is equivalent to normalization using urine creatinine levels. Burton et al., Clin Chim Acta, 435:42-47 (2014). Specimens were than stored in a freezer in the clinic at −20° C. for a period of 4 to 6 hours and were then taken to the proteomics laboratory where they were stored at −80° C. Transit time from the clinic site to the laboratory was less than 30 minutes.

Choline detection was accomplished using the Ch/ACh assay kit (ab65345; Abcam, Cambridge, Mass.) using the colorimetric method following the manufacturer's instructions. Urine samples were centrifuged at 2000× gravity for 30 seconds to pellet any precipitate, and 50 μL undiluted urine was mixed with an equal volume of Ch reaction mix in duplicate wells in 96-well flat-bottom acrylic microtiter plates (#3635; Corning, Kennebunk, Me.). Background readings were obtained by mixing sample with assay buffer only.

Plates were incubated in the dark for 30 minutes at room temperature, and an optical density with a wavelength of 570 nm (OD₅₇₀) was measured with a microplate reader (accuSkan FC, with Skanit RE 4.0 software; Thermo Fisher). Free Ch was measured by comparing OD values to a Ch standard curve. Two additional low-end standards (0.5 and 0.25 nmol/well) were included with the kit's suggested standards (5-0 nmol/well).

To measure acetylcholine, acetylcholinesterase was added to the reaction mix in additional wells on the same plate to convert it to Ch and determine total Ch (free Ch+acetylcholine). Acetylcholine values were obtained by subtracting the free Ch value from the total Ch value. Although the Abcam assay kit is capable of detecting urinary acetylcholine levels, the concentration of acetylcholine in our study sample was below the sensitivity of the test; therefore, this measurement was not included in the analysis.

The complete disclosure of all patents, patent applications, and publications, and electronically available material cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method of diagnosing the risk that a subject has or has an increased risk of developing overactive bladder syndrome, comprising detecting the levels of choline and/or acetylcholine in a biological sample from the subject, comparing the detected levels to control values, and characterizing the subject as having an increased risk of having or developing overactive bladder syndrome if the choline and/or acetylcholine values are higher than the control values.

2. The method of claim 1, wherein the subject is a human female.

3. The method of claim 2, wherein the human female is post-menopausal.

4. The method of claim 1, wherein the biological sample is urine.

5. The method of claim 1, further comprising obtaining the biological sample from a subject.

6. A method of treating a subject for overactive bladder syndrome, comprising the step of determining the level of acetylcholine in a biological sample from a subject having overactive bladder syndrome, and treating the subject with a therapeutically effective amount of an anticholinergic agent if the biological sample from the subject is has a higher acetylcholine level than a control value.

7. The method of claim 6, wherein the subject is a human female.

8. The method of claim 7, wherein the human female is post-menopausal.

9. The method of claim 6, wherein the biological sample is urine.

10. The method of claim 6, further comprising obtaining the biological sample from a subject.

11. The method of claim 6, wherein the overactive bladder syndrome is treated using anti-cholinergic therapy.

12. A kit for determining choline and/or acetylcholine levels in a subject, comprising a reagent for detecting the level of choline and/or acetylcholine in a biological sample obtained from the subject, and means for signaling the levels of choline and/or acetylcholine detected in the biological sample.

13. The kit of claim 12, wherein the biological sample is urine.

14. The kit of claim 12, wherein the kit provides a point-of-care diagnosis.

15. The kit of claim 12, wherein the reagent for detecting the level of choline and/or acetylcholine is on a dip stick.

16. The kit of claim 12, wherein the kit detects acetylcholine.

17. The kit of claim 12, further comprising instructions for using the kit to treat or diagnose a subject for overactive bladder syndrome.

18. The kit of claim 12, further comprising a package to hold the components of the kit.
